(19) United States
(12) Patent Application Publication
(10) Pub. No.: US 2025/0332083 A1
(43) Pub. Date: Oct. 30, 2025

(54) SKIN CARE COMPOSITION COMPRISING PEPTIDE DERIVATIVE AND FATTY ACID, AND METHOD OF USING THE SAME

(71) Applicant: The Procter & Gamble Company, Cincinnati, OH (US)

(72) Inventors: Lu Zhang, Singapore (SG); Peter Brendan Styczynski, West Chester, OH (US); Chuiying Li, Mason, OH (US)

(21) Appl. No.: 19/188,162

(22) Filed: Apr. 24, 2025

Related U.S. Application Data
(60) Provisional application No. 63/638,008, filed on Apr. 24, 2024.

(51) Int. Cl. A61K 8/64; A61K 8/36; A61Q 19/00

(52) U.S. Cl. CPC A61K 8/64; A61K 8/361; A61Q 19/00

(57) ABSTRACT

A cosmetic skin care composition having a peptide derivative, a fatty acid having from about 8 to about 22 carbon atoms, and a dermatologically acceptable carrier. The peptide derivative has a formula: Rc-R₁-Gly-Pro-Gln-Gly-R₂, where Rc is chosen from hydroxycinnamic acid, its derivative, and mixtures thereof, where R₁ and R₂ are respectively from about 0 to about 8 amino acids, and wherein a total of R₁ and R₂ is 8 or less amino acids; The composition provides improved penetration of such peptide derivatives to skin.

### FIELD

The present disclosure is directed to a cosmetic skin care composition, comprising: (a) a peptide derivative having a formula: Rc-R₁-Gly-Pro-Gln-Gly-R₂, wherein Rc is selected from the group of hydroxycinnamoyl, its derivative, and mixtures thereof, wherein R₁ and R₂ are respectively from about 0 to about 8 amino acids, and wherein a total of R₁ and R₂ is 8 or less amino acids; (b) a fatty acid having from about 8 to about 22 carbon atoms; and (c) a dermatologically acceptable carrier. The composition provides improved penetration of such peptide derivatives to skin.

### BACKGROUND

Skin is the first line of defense against environmental insults that would otherwise damage sensitive underlying tissue and organs. Additionally, skin plays a key role in a person's physical appearance. Not surprisingly, most people would like to have heathy, younger looking skin. Unfortunately for some people, the tell-tale signs of aging such as thinning skin, wrinkles, and age spots are an undesirable reminder of the disappearance of youth. The desire for healthy, younger looking skin has led to the development of numerous skin care products marketed to treat the various skin conditions associated with aging and unhealthy skin. These skin care products typically include one or more active ingredients for treating a skin condition of interest.

Peptides and peptide derivatives are commonly used in skin care products as active ingredients. For example, KR10-1942844 B1 from AEKYUNG discloses a cream formulation in Example 7 in Table 15, comprising 0.01% of peptide (Gallic-DA-NH2) and 8.0% stearic acid, and other ingredients. Another example could be US 20130079287 A from BIO FD&C, disclosing Nutrition Lotion in Example 2 in Table 8, which comprises 1% of Aqueous Solution of Caffeoyl Peptide Derivative (100 ppm) of Example 1 and 1.5% of stearic acid and other ingredients. Also, WO2023077338A1 discloses a hexapeptide derivative obtained by covalently binding caffeic acid and gallic acid, and its cosmetic composition or pharmaceutical composition and application.

However, it has been found by the present inventors that some peptide derivatives are difficult to penetrate into skin.

Thus, there remains a need to provide skin care composition which provides improved penetration of such peptide derivatives to skin.

### SUMMARY

Disclosed herein is a cosmetic skin care composition, comprising:
(a) a peptide derivative having a formula: Rc-R₁-Gly-Pro-Gln-Gly-R₂,
   wherein Rc is selected from the group of hydroxycinnamoyl, its derivative, and mixtures thereof,
   wherein R₁ and R₂ are respectively from about 0 to about 8 amino acids, and wherein a total of R₁ and R₂ is 8 or less amino acids;
(b) a fatty acid having from about 8 to about 22 carbon atoms; and
(c) a dermatologically acceptable carrier.

The composition provides improved penetration of such peptide derivatives to skin.

It has been found by the present inventors that some peptide derivatives, especially peptide derivatives such as Caffeoyl Hexapeptide 9, are difficult to penetrate into skin, especially from cream composition containing a higher amount of oily components.

It has been surprisingly found by the present inventors that, by the use of specific fatty acids together with such peptide derivatives, especially at their preferred levels and a ratio, the skin care composition provides improved penetration of such peptide derivatives to skin.

### DETAILED DESCRIPTION

Reference herein to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

In all embodiments, all ingredient percentages are based on the weight of the cosmetic composition, unless specifically stated otherwise. All ratios are weight ratios, unless specifically stated otherwise. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive and combinable to form narrower ranges not explicitly disclosed. For example, delineated upper and lower range limits are interchangeable to create further ranges.

The compositions can comprise, consist essentially of, or consist of, the essential components as well as optional ingredients described herein. As used herein, “consisting essentially of” means that the composition or component may only include additional ingredients that do not materially alter the basic and novel characteristics of the claimed composition or method. As used in the description and the appended claims, the singular forms “a”, “an”, and “the” are intended to include the plural forms as well, unless the context clearly indicates otherwise.

### Definitions

“About” modifies a particular value by referring to a range equal to plus or minus twenty percent (+/−20%) or less (e.g., less than 15%, 10%, or even less than 5%) of the stated value.

“Apply” or “application”, as used in reference to a composition, means to apply or spread the compositions onto a human skin surface such as the epidermis.

“Cosmetic composition” means a composition comprising a cosmetic agent and intended for non-therapeutic (i.e., medical) use. Examples of cosmetic compositions include color cosmetics (e.g., foundations, lipsticks, concealers, and mascaras), skin care compositions (e.g., moisturizers and sunscreens), personal care compositions (e.g., rinse-off and leave on body washes and soaps), hair care compositions (e.g., shampoos and conditioners).

“Effective amount” means an amount of a compound or composition sufficient to significantly induce a positive benefit to keratinous tissue over the course of a treatment period. The positive benefit may be a health, appearance, and/or feel benefit, including, independently or in combination, the benefits disclosed herein.

“Skin care” means regulating and/or improving a skin condition (e.g., skin health, appearance, or texture/feel). Some nonlimiting examples of improving a skin condition include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin.

“Skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

“Skin care composition” means a composition that includes a skin care active and regulates and/or improves skin condition.

“Treatment period,” as used herein, means the length of time and/or frequency that a material or composition is applied to a target skin surface.

### Skin Care Composition

The present cosmetic skin care composition, comprises:
a) a peptide derivative having a formula: Rc-R₁-Gly-Pro-Gln-Gly-R₂,
   wherein Rc is selected from the group of hydroxycinnamoyl, its derivative, and mixtures thereof,
   wherein R₁ and R₂ are respectively from about 0 to about 8 amino acids, and wherein a total of R₁ and R₂ is 8 or less amino acids;
b) a fatty acid having from about 8 to about 22 carbon atoms; and
c) a dermatologically acceptable carrier.

In the composition, the peptide and the fatty acid are present at a ratio of preferably from about 1:5 to about 1:1000, more preferably from about 1:10 to about 1:100. Still more preferably from about 1:10 to about 1:50.

The skin care compositions herein may be cosmetic compositions, pharmaceutical compositions, or cosmeceutical compositions, and may be provided in various product forms, including, but not limited to, solutions, suspensions, lotions, creams, gels, serums, essences, toners, sticks, sprays, aerosols, ointments, cleansing liquid washes and solid bars, pastes, foams, mousses, shaving creams, wipes, strips, patches, electrically-powered patches, hydrogels, film-forming products, facial and skin masks (with and without insoluble sheet), make-up such as foundations, eye liners, and eye shadows, and the like. In some instances, the composition form may follow from the particular dermatologically acceptable carrier chosen. For example, the composition (and carrier) may be provided in the form of an emulsion (e.g., water-in-oil, oil-in-water, or water-in-oil-in water) or an aqueous dispersion. Preferably, the composition is in the form of oil-in-water emulsion, and/or in the form of cream containing a higher amount of oily components, in view of enjoying the benefit of improved penetration of the peptide derivative while providing more hydration feeling compared to, for example, essence and/or lotion containing reduced amount of oily components. “Cream containing a higher amount of oily components” herein means, cream composition containing 3% or more, preferably 5% or more, more preferably 7% or more of the oily components, wherein the oily components are, for example, solid, paste, and/or liquid oily components selected from the group consisting of fatty alcohol, ester oils, hydrocarbon oils, vegetable oils and mixtures thereof. Fatty acids and Silicone oils are not included in the oily components herein.

The compositions herein may be prepared by conventional methods of making topical skin care compositions. Such methods typically involve mixing of the ingredients in one or more steps to a relatively uniform state, with or without heating, cooling, application of vacuum, and the like. The compositions are preferably prepared such as to optimize stability (physical stability, chemical stability, photostability) and/or delivery of the active materials. This optimization may include appropriate pH (e.g., less than 7), exclusion of materials that can complex with the active agent and thus negatively impact stability or delivery (e.g., exclusion of contaminating iron), use of approaches to prevent complex formation (e.g., appropriate dispersing agents or dual compartment packaging), use of appropriate photostability approaches (e.g., incorporation of sunscreen/sunblock, use of opaque packaging), etc.

### Peptide Derivative

The peptide derivative useful herein has a formula: Rc-R₁-Gly-Pro-Gln-Gly-R₂,
wherein Rc is selected from the group of hydroxycinnamoyl, its derivative, and mixtures thereof, wherein R₁ and R₂ are respectively from about 0 to about 8 amino acids, alternatively from about 0 to about 4 amino acids. A total of R₁ and R₂ is 8 or less amino acids, preferably from about 0 to 4 amino acids. The peptide derivative is obtained by covalent binding between the peptide and hydroxycinnamic acid, its derivative, and mixtures thereof.

R₁ can be zero and R₂ can be from about 0 to 8 amino acids, alternatively R₁ can be zero and R₂ can be from about 0 to 4 amino acids, alternatively R₁ can be zero and R₂ can be from about 1 to 7 amino acids, alternatively R₁ can be zero and R₂ can be from about 1 to 5 amino acids. R₂ can be X or X-Gln, wherein X can be Pro, Ile-Ala-Ser, or Pro-Ala-Ser.

R₁ and/or R₂ can be in the formula in an amount greater than 0 and less than 8.

Rc is selected from the group consisting of caffeoyl (4-Hydroxycinnamoyl), para-coumaroyl feruloyl and mixtures thereof, i.e., the peptide derivative is obtained by covalent binding between the peptide and acid selected from the group consisting of caffeic acid, para-coumaric acid, ferulic acid.

Highly preferred peptide derivative is Caffeoyl Hexapeptide 9, wherein Rc is caffeoyl, R₁ is zero and R₂ is Pro-Gln in the above formula.

The peptide is present at a level of preferably from about 0.0001% to about 5%, more preferably from about 0.001% to about 1%, still more preferably from about 0.003% to about 0.5%, by weight of the composition.

### Fatty Acid

The fatty acids useful herein are those having from about 8 to about 22 carbon atoms. Preferably, the fatty acids are those selected from the group consisting of stearic acid, palmitic acid, and mixtures thereof. More preferably, the fatty acids are a mixture of stearic acid and palmitic acid, and still more preferably, the mixture of stearic acid and palmitic acid at a weight ratio of from about 3:7 to about 7:3.

The fatty acid is present at a level of, preferably from about 0.01% to about 10%, more preferably from about 0.05% to about 5%, still more preferably from about 0.05% to about 1%, by weight of the composition.

### Substantially Free of Other Fatty Acids

In the composition, it may be preferred that the composition is substantially free of other fatty acids than those described above. It is not being bound to any theory, but it may be thought that fatty acids having longer alkyl chain may affect the penetration of the peptide derivatives and/or may provide undesired interfere with other ingredients, and fatty acids having shorter alkyl chain may not have any benefit to include. “Substantially free of other fatty acids” herein means that the composition contains 0.008% or less, preferably 0.005% or less of such other fatty acids. More preferably, the composition is free of such other fatty acids, i.e., contains 0% of such other fatty acids.

### Dermatologically Acceptable Carrier

The compositions herein include a dermatologically acceptable carrier (which may be referred to as a “carrier”). The phrase “dermatologically acceptable carrier” means that the carrier is suitable for topical application to the keratinous tissue, has good aesthetic properties, is compatible with the actives in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight of the composition.

The carrier can be in a wide variety of forms. In some instances, the solubility or dispersibility of the components (e.g., extracts, sunscreen active, additional components) may dictate the form and character of the carrier. Non-limiting examples include simple solutions (e.g., aqueous or anhydrous), dispersions, emulsions, and solid forms (e.g., gels, sticks, flowable solids, or amorphous materials). In some instances, the dermatologically acceptable carrier is in the form of an emulsion that has a continuous aqueous phase (e.g., an oil-in-water or water-in-oil-in-water emulsion) or a continuous oil phase (e.g., water-in-oil or oil-in-water-in-oil emulsion). The oil phase of the emulsion may include silicone oils, non-silicone oils such as hydrocarbon oils, esters, ethers, and mixtures thereof. The aqueous phase may include water and water-soluble ingredients (e.g., water-soluble moisturizing agents, conditioning agents, anti-microbials, humectants and/or other skin care actives). In some instances, the aqueous phase may include components other than water, including but not limited to water-soluble moisturizing agents, conditioning agents, anti-microbials, humectants and/or other water-soluble skin care actives. In some instances, the non-water component of the composition comprises a humectant such as glycerin and/or other polyol(s).

In some instances, the compositions herein are in the form of an oil-in-water (“O/W”) emulsion that provides a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 30% by weight of composition (e.g., 1% to 20%, 2% to 15%, 3% to 12%, 4% to 10%, or even 5% to 8%) to help avoid some of the undesirable feel effects of oily compositions. The oil phase may include one or more volatile and/or non-volatile oils (e.g., botanical oils, silicone oils, and/or hydrocarbon oils). Some nonlimiting examples of oils that may be suitable for use in the present compositions are disclosed in U.S. Pat. No. 9,446,265 and U.S. Publication No. 2015/0196464.

The carrier may contain one or more dermatologically acceptable diluents. As used herein, “diluent” refers to materials in which the skin care actives herein can be dispersed, dissolved, or otherwise incorporated. Some non-limiting examples of hydrophilic diluents include water, organic hydrophilic diluents such as lower monovalent alcohols (e.g., C₁-C₄) and low molecular weight glycols and polyols, including propylene glycol, polyethylene glycol (e.g., molecular weight of 200 to 600 g/Mole), polypropylene glycol (e.g., molecular weight of 425 to 2025 g/mole), glycerol, butylene glycol, 1,2,4-butanetriol, sorbitol esters. 1,2,6-hexanetriol, ethanol, isopropanol, sorbitol esters, butanediol, ether propanol, ethoxylated ethers, propoxylated ethers and combinations thereof.

### Vitamin B₃ Compound

The compositions herein may optionally include a safe and effective amount of a vitamin B₃ compound. In some instances, the present compositions may contain 0.01% to 10%, by weight, of the vitamin B₃ compound, based on the weight or volume of the composition (e.g., 0.1% to 10%, 0.5% to 5%, or even 1% to %).

As used herein, “vitamin B₃ compound” means a compound having the formula:

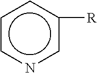

Where: R is CONH₂ (i.e., niacinamide), COOH (i.e., nicotinic acid) or CH₂OH (i.e., nicotinyl alcohol); derivatives thereof; and salts of any of the foregoing.

Exemplary derivatives of vitamin B₃ compounds include nicotinic acid esters, including non-vasodilating esters of nicotinic acid (e.g., tocopheryl nicotinate, myristyl nicotinate) nicotinamide riboside, nicotinyl amino acids, nicotinyl alcohol esters of carboxylic acids, nicotinic acid N-oxide, and niacinamide N-oxide.

### Dipeptide

The compositions herein may include a safe and effective amount of the palmitoylated diopeptide, pal-KT (INCI: Palmitoyl Dipeptide-7). In some instances, pal-KT may be present in the present compositions at 0.0001% to 3% (e.g., 0.0001% to 2%, 0.01% to 1% or 0.1% to 0.5%). Pal-KT is available as Palestrina® from Sederma (France).

### Tetrapeptide

The compositions herein may include a safe and effective amount of the acetylated tetrapeptide, ac-PPYL (INCI: Acetyl Tetrapeptide-11). In some instances, ac-PPYL may be present in the present composition at 0.0001% to 3% (e.g., 0.001% to 2%, 0.01% to 1% or 0.1% to 0.5%). Ac-PPYL is available as SYNIORAGE from BASF Care Creations (New Jersey).

### Pentapeptide

The compositions herein may include a safe and effective amount of the palmitoylated penta peptide, pal-KTTKS (INCI: Palmitoyl Pentapeptide-4). In some instances, pal-KTTKS may be present in the present compositions at 0.00005% to 3% (e.g., 0.00005% to 2%, 0.01% to 1% or 0.1% to 0.5%).

### Conditioning Agents

The compositions herein may include 0.1% to 50% by weight of a conditioning agent (e.g., 0.5% to 30%, 1% to 20%, or even 2% to 15%). Adding a conditioning agent can help provide the composition with desirable feel properties (e.g., a silky, lubricious feel upon application). Some non-limiting examples of conditioning agents include, hydrocarbon oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, lanolin, wax esters, beeswax derivatives, sterols and phospholipids, salts, isomers and derivatives thereof, and combinations thereof. Particularly suitable examples of conditioning agents include volatile or non-volatile silicone fluids such as dimethicone copolyol, dimethylpolysiloxane, diethylpolysiloxane, mixed C1-30 alkyl polysiloxanes, phenyl dimethicone, dimethiconol, dimethicone, dimethiconol, silicone crosspolymers, and combinations thereof. Dimethicone may be especially suitable, since some consumers associate the feel properties provided by certain dimethicone fluids with good moisturization. Other examples of silicone fluids that may be suitable for use as conditioning agents are described in U.S. Pat. No. 5,011,681.

### Rheology Modifiers

The compositions herein may include 0.1% to 5% of a rheology modifier (e.g., thickening agent) to provide the composition with suitable rheological and skin feels properties. Some non-limiting examples of thickening agents include crosslinked polyacrylate polymers, polyacrylamide polymers, polysaccharides, gums and mixtures thereof. In a particularly suitable example, the composition may include a superabsorbent polymer thickening agent such as sodium polyacrylate, starch grafted sodium polyacrylate, or a combination of these. Some non-limiting examples of superabsorbent polymer thickeners are described in, for example, U.S. Pat. No. 9,795,552.

Some consumers find compositions that use silicone fluids as conditioning agents to be undesirably greasy or heavy feeling. Thus, it may be desirable to provide a composition that is free of or substantially free of silicone fluid. It may also be desirable to tailor a superabsorbent polymer thickener to provide the composition with a light, airy feel, for example, by adjusting the amount of water in the composition, the water:oil ratio (e.g., 12:1 to 1:1), and/or the ratio of water to thickener or oil to thickener.

### Emulsifiers

When the dermatologically acceptable carrier is in the form of an emulsion, it may be desirable to include an emulsifier to provide a stable composition (e.g., does not phase separate), When included, the emulsifier may be present at an amount of 0.1% to 10% (e.g., 1% to 5%, or 2%-4%). Emulsifiers may be nonionic, anionic or cationic. Some non-limiting examples of emulsifiers that may be suitable for use herein are disclosed in U.S. Pat. Nos. 3,755,560; 4,421,769; and McCutcheon's Detergents and Emulsifiers, North American Edition, pages 317-324 (1986).

### Other Optional Ingredients

The present composition may optionally include one or more additional ingredients commonly used in cosmetic compositions (e.g., colorants, skin care actives, anti-inflammatory agents, sunscreen agents, emulsifiers, buffers, rheology modifiers, combinations of these and the like), provided that the additional ingredients do not undesirably alter the skin health or appearance benefits provided by the present compositions. The additional ingredients, when incorporated into the composition, should be suitable for use in contact with human skin tissue without undue toxicity, incompatibility, instability, allergic response, and the like. Some nonlimiting examples of additional actives include vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunless tanning agents, lubricants, anti-acne actives, anti-cellulite actives, chelating agents, anti-wrinkle actives, anti-atrophy actives, phytosterols and/or plant hormones, N-acyl amino acid compounds, antimicrobials, and antifungals. Other non-limiting examples of additional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/00092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

When including optional ingredients in the compositions herein, it may be desirable to select ingredients that do not form complexes or otherwise undesirably interact with other ingredients in the composition, especially pH sensitive ingredients like niacinamide, salicylates and peptides. When present, the optional ingredients may be included at amounts of from 0.0001% to 50%; from 0.001% to 20%; or even from 0.01% to 10% (e.g., 50%, 40%, 30%, 20%, 10%, 5%, 4%, 3%, 2%, 1%, 0.5% or 0.1%), by weight of the composition.

### Method of Use

The present disclosure also relates to a non-therapeutic method of treating skin, comprising:
a) identifying a target portion of skin where treatment is desired; and
b) applying the skin care composition to the target portion of skin over the course of a treatment period.

The method improves penetration of the peptide derivative into skin.

The target portion of skin may be on a facial skin surface such as the forehead, perioral, chin, periorbital, nose, and/or cheek) or another part of the body (e.g., hands, arms, legs, back, chest). The person or target portion of skin in need of treatment may be one that exhibits a telltale sign of aging skin (e.g., fine lines, wrinkles, hyperpigmented spots). In some instances, a target portion of skin may not exhibit a sign of skin aging, but a user may still wish to treat the portion of skin if it is one that is known to exhibit visible signs of aging (e.g., skin that is exposed to the sun). In this way, the present methods and compositions may be used prophylactically to help delay the visible signs of skin aging.

The composition may be applied to a target portion of skin and, if desired, to the surrounding skin at least once a day, twice a day, or on a more frequent daily basis, during a treatment period. When applied twice daily, the first and second applications are separated by at least 1 to 12 hours. Typically, the composition is applied in the morning and/or in the evening before bed. The treatment period herein is ideally of sufficient time for the composition to improve the appearance of the skin. The treatment period may last for at least 1 week (e.g., about 2 weeks, 4 weeks, 8 weeks, or even 12 weeks). In some instances, the treatment period will extend over multiple months (i.e., 3-12 months). In some instances, the composition may be applied most days of the week (e.g., at least 4, 5 or 6 days a week), at least once a day or even twice a day during a treatment period of at least 2 weeks, 4 weeks, 8 weeks, or 12 weeks.

The step of applying the composition may be accomplished by localized application. In reference to application of the composition, the terms “localized”, “local”, or “locally” mean that the composition is delivered to the targeted area (e.g., a wrinkle or portion thereof) while minimizing delivery to skin surfaces where treatment is not desired. The composition may be applied and lightly massaged into an area of skin. The form of the composition or the dermatologically acceptable carrier should be selected to facilitate localized application. While certain embodiments herein contemplate applying a composition locally to an area, it will be appreciated that compositions herein can be applied more generally or broadly to one or more skin surfaces. In certain embodiments, the compositions herein may be used as part of a multi-step beauty regimen, wherein the present composition may be applied before and/or after one or more other compositions.

### Examples

The following are examples of skin care compositions which are suitably used as oil-in-water emulsion cream compositions.

These compositions are made by blending the A phase components with a suitable mixer (e.g., Tekmar RW20DZM or equivalent) and heating to a temperature of 50-80° C. and maintaining the temperature while stirring. Separately, the B phase components are blended with a suitable mixer and heated to 60-75° C., while maintaining temperature during mixing. Phase B is added to Phase A while mixing well to form an oil-in-water (O/W) emulsion. The emulsion is then milled using a suitable mill (e.g., Tekmar T-25 or equivalent) for 5 minutes. When the emulsion is at 45-60° C., phase C is added while continuing to mix. At 40° C., the ingredients of phase D and E are added to the emulsion. The emulsion is then milled for 5 minutes to provide a uniform composition.

Detailed compositional breakdown of these compositions is listed as follows (see Tables 1 and 3).

Ex. A through Ex. O in Tables 1, 3 and 4 are the examples of the composition of the present disclosure. The examples of the composition of the present disclosure provide improved penetration of such peptide derivatives to skin. Such benefit, especially improved penetration can be also understood by the comparison of Ex. A to the comparative example in Table 2. In more detail, Ex. A which contains 0.005% of Caffeoyl Hexapeptide-9 and 0.1% of fatty acid shows improved penetration into skin (Please refer to “Fold Increase of Ex. A compared to Comparative example” in Table 2), compared to the comparative example which also contains 0.005% of Caffeoyl Hexapeptide-9 but does not contain any fatty acid.

TABLE 1

| | Comparative | |
|---|---|---|
| | example | Ex. A |
| Component | % w/w | % w/w |
| Phase A | | |
| Water Purified | QS | QS |
| Sodium Hydroxide | 0.01 | 0.01 |
| Panthenol | 1.00 | 1.00 |
| Glycerine 99% | 8.00 | 8.00 |
| Niacinamide | 5.00 | 5.00 |
| Pentylene glycol | 3.00 | 3.00 |
| Disodium EDTA | 0.05 | 0.05 |
| Polyacrylamide & C13-14 Isoparaffin | 2.00 | 2.00 |
| Lipobrite *1 | 0.67 | 0.67 |
| Phase B | | |
| Cetyl Alcohol | 1.00 | 1.00 |
| Stearyl Alcohol | 1.25 | 1.25 |
| Behenyl Alcohol | 0.75 | 0.75 |
| Stearic acid and Palmitic acid blend (40/60) | — | 0.10 |
| PEG 100 Stearate | 0.10 | 0.10 |
| Cetearyl Glucoside & Cetearyl alcohol | 0.20 | 0.20 |
| Isopropyl Isostearate | 1.33 | 1.33 |
| Isohexadecane | 3.00 | 3.00 |
| Butyrospermum Parkii (Shea Butter) | 1.00 | 1.00 |
| Phase C | | |
| Symsave H | 0.15 | 0.15 |
| Phenoxyethanol | 0.25 | 0.25 |
| Caffeoyl Hexapeptide-9 | 0.005 | 0.005 |

*1 LIPOBRITE available from Vantage (15% 4-HCA, 85% PEG-4)

TABLE 2

| | | | | | Fold Increase of Ex. A compared |
|---|---|---|---|---|---|
| | | Comparative | | P | to Comparative example, |
| | | example | Ex. A | Value | in CH-9 Skin Penetration |
| Franz Cell | Skin Pen-Epidermis | 6.60 | 29.61 | 0.0001 | 4.49 |
| (in vitro) | (% Applied Dose) | | | | |
| Skin | Skin Pen-Dermis | 4.09 | 7.37 | 0.001 | 1.80 |
| Penetration | (% Applied Dose) | | | | |
| Results | Skin Pen-Total | 20.23 | 43.03 | 0.0009 | 2.12 |
| | (% Applied Dose) | | | | |

TABLE 3

| | % w/w | | | | | | |
|---|---|---|---|---|---|---|---|
| Component | A | B | C | D | E | F | G |
| Phase A | | | | | | | |
| Water Purified | QS | QS | QS | QS | QS | QS | QS |
| Panthenol | 1.00 | 0.50 | 1.00 | 1.00 | 1.00 | 1.00 | 0.50 |
| Glycerine 99% | 8.00 | 8.00 | 8.00 | 8.00 | 8.00 | 8.00 | 8.00 |
| Niacinamide | 5.00 | 5.00 | 5.00 | 5.00 | 5.00 | 5.00 | 5.00 |
| Pentylene glycol | 3.00 | 3.00 | 3.00 | 3.00 | 3.00 | 3.00 | 3.00 |
| Disodium EDTA | 0.05 | 0.05 | 0.05 | 0.05 | 0.05 | 0.05 | 0.05 |
| Polyacrylamide & | 2.00 | 1.0 | 3.5 | 3.5 | 2.5 | 3 | 2.00 |
| C13-14 | | | | | | | |
| Isoparaffin | | | | | | | |
| Phase B | | | | | | | |
| Cetyl Alcohol | 1.00 | 0.40 | 0.50 | 1.00 | 0.70 | 0.80 | 1.00 |
| Stearyl Alcohol | 1.25 | 0.50 | 1.00 | 1.25 | 0.80 | 1.00 | 1.20 |
| Behenyl Alcohol | 0.75 | 0.30 | 1.00 | 0.75 | 0.90 | 0.60 | 0.75 |
| Stearic acid and | 0.10 | 0.05 | 0.02 | 0.20 | 0.10 | 0.15 | 0.10 |
| Palmitic acid | | | | | | | |
| blend (50/50) | | | | | | | |
| PEG 100 Stearate | 0.10 | 0.10 | 0.10 | 0.10 | 0.10 | 0.10 | 0.10 |
| Cetearyl | 0.20 | 0.20 | 0.20 | 0.20 | 0.20 | 0.20 | 0.20 |
| Glucoside & | | | | | | | |
| Cetearyl alcohol | | | | | | | |
| Isopropyl | 1.33 | 1.33 | 1.33 | 1.33 | 1.33 | 1.33 | 1.33 |
| Isostearate | | | | | | | |
| Isohexadecane | 3.00 | 3.00 | 3.00 | 3.00 | 3.00 | 3.00 | 3.00 |
| Butyrospermum | 1.00 | 1.00 | 1.00 | 1.00 | 1.00 | 1.00 | 1.00 |
| Parkii (Shea | | | | | | | |
| Butter) | | | | | | | |
| Phase C | | | | | | | |
| Symsave H | 0.15 | 0.15 | 0.15 | 0.15 | 0.15 | — | 0.15 |
| Phenoxyethanol | 0.25 | 0.25 | 0.25 | 0.25 | 0.25 | 0.50 | 0.25 |
| Caffeoyl | 0.005 | 0.005 | 0.0065 | 0.01 | 0.05 | 0.005 | 0.0005 |
| Hexapeptide | | | | | | | |

TABLE 4

| | % w/w | | | | | | | |
|---|---|---|---|---|---|---|---|---|
| Component | H | I | J | K | L | M | N | O |
| Phase A | | | | | | | | |
| Water Purified | QS | QS | QS | QS | QS | QS | QS | QS |
| Panthenol | 0.50 | 0.50 | 0.50 | 0.50 | 0.50 | 0.50 | 0.50 | 0.50 |
| Glycerine 99% | 8.00 | 8.00 | 8.00 | 8.00 | 8.00 | 8.00 | 8.00 | 8.00 |
| Niacinamide | 5.00 | 5.00 | 5.00 | 5.00 | 5.00 | 5.00 | 5.00 | 5.00 |
| Pentylene glycol | 3.00 | 3.00 | 3.00 | 3.00 | 3.00 | 3.00 | 3.00 | 3.00 |
| Disodium EDTA | 0.05 | 0.05 | 0.05 | 0.05 | 0.05 | 0.05 | 0.05 | 0.05 |
| Polyacrylamide & | 2.50 | 2.50 | 2.00 | 2.00 | 2.50 | 2.50 | 2.50 | 2.50 |
| C13-14 | | | | | | | | |
| Isoparaffin | | | | | | | | |
| Phase B | | | | | | | | |
| Cetyl Alcohol | 1.00 | 1.00 | 0.70 | 1.00 | 1.00 | 1.00 | 1.00 | 1.00 |
| Stearyl Alcohol | 1.25 | 1.25 | 0.80 | 1.20 | 1.25 | 1.25 | 1.25 | 1.25 |
| Behenyl Alcohol | 0.75 | 0.75 | 0.90 | 0.75 | 0.75 | 0.75 | 0.75 | 0.75 |
| Stearic acid | — | — | — | 0.10 | — | — | — | — |
| Palmitic acid | 0.02 | 0.05 | 0.10 | — | — | — | — | — |
| Myristic acid (c14) | — | — | — | — | — | — | 0.1 | — |
| Arachidic Acid (c20) | — | — | — | — | — | — | — | 0.1 |
| caprylic acid (c8) | — | — | — | — | 0.1 | — | — | — |
| Docosanoic acid (c22) | — | — | — | — | — | 0.1 | — | — |
| PEG 100 Stearate | 0.10 | 0.10 | 0.10 | 0.10 | 0.10 | 0.10 | 0.10 | 0.10 |
| Cetearyl | 0.20 | 0.20 | 0.20 | 0.20 | 0.20 | 0.20 | 0.20 | 0.20 |
| Glucoside & | | | | | | | | |
| Cetearyl alcohol | | | | | | | | |
| Isopropyl | 1.33 | 1.33 | — | 1.33 | 1.33 | 1.33 | 1.33 | 1.33 |
| Isostearate | | | | | | | | |
| Isohexadecane | 3.00 | — | 3.00 | 3.00 | 3.00 | 3.00 | 3.00 | 3.00 |
| Butyrospermum | — | 1.00 | 1.00 | 1.00 | 1.00 | 1.00 | 1.00 | 1.00 |
| Parkii (Shea | | | | | | | | |
| Butter) | | | | | | | | |
| Phase C | | | | | | | | |
| Symsave H | — | — | 0.15 | 0.15 | 0.15 | 0.15 | 0.15 | 0.15 |
| Phenoxyethanol | 0.50 | 0.50 | 0.25 | 0.25 | 0.25 | 0.25 | 0.25 | 0.25 |
| Caffeoyl | 0.0001 | 0.0010 | 0.0005 | 0.005 | 0.005 | 0.005 | 0.005 | 0.005 |
| Hexapeptide | | | | | | | | |

### Skin Penetration Method (Franz Cell)

In vitro skin penetration of actives, such as niacinamide, from topically applied formulations can be determined using the Franz diffusion cell assay (Franz, T. J. Percutaneous absorption. On the relevance of in vitro data. J. Invest. Dermatol. 64: 190-195, 1975; Franz, et al. The use of excised human skin to assess the bioequivalence of topical products. Skin Pharmacol. Physiol. 22: 276-286, 2009). The Franz diffusion cell assay is widely used in the skin care industry for assessment of skin penetration and for the dermal absorption safety assessments.

Skin samples are prepared from split-thickness human cadaver skin that is thawed at ambient conditions, cut into appropriately sized sections, and mounted in standard static Franz-type diffusion cells (0.79 cm² surface area) maintained at 37° C. Approximately 5 ml of a receptor solution is placed in the receptor compartments at the bottom of each cell to collect any peptide or other material of interest the penetrates through the entire skin sample. The receptor solution is phosphate buffered saline (PBS-pH 7.4) that includes 1% polysorbate-20 and 0.02% sodium azide. The skin samples are equilibrated for two hours. Each treatment group has 6 replicates.

To prepare the test compositions, aliquots of the test compositions are spiked with ³H-Caffeoyl Hexapeptide-9 (3 μCi per 300 mg aliquot). The test composition aliquots are mixed and assayed for total radioactivity in triplicate using ULTIMA GOLD brand liquid scintillation cocktail (PerkinElmer, Boston) or equivalent and a suitable liquid scintillation counter (e.g., TRI-CARB 2500 TR brand liquid scintillation analyzer available from PerkinElmer).

The skin samples are topically dosed with 5 μL of the test composition using a positive displacement pipette. The product is gently spread over the surface of the skin (˜0.79 cm²) using a glass rod. At the end of the test (i.e., 4 hours after dosing) the receptor solution is collected and the surface of each skin sample is wiped two times with Whatman filter paper soaked with PBS/Tween 20 and once with 70%/30% ethanol/water to remove unabsorbed (residual) product. The epidermis is separated from the dermis by dissection and then the epidermis and dermis sections are dissolved in 0.50-1.25 mL SOLUENE-350 (available from PerkinElmer) at 60° C. overnight. Caffeoyl Hexapeptide-9 skin penetration is quantitated using liquid scintillation counting as described above. Scintillation counting is performed on the epidermis sample, the dermis sample, and the receptor solution, as well as the filter papers used to wipe the skin surface. The amount of epidermal skin penetration is the total amount of radioactivity measured from the epidermis sample (including stratum corneum). Total penetration is the sum of the radioactivity measured from the epidermis sample, the dermis sample, and the receptor compartment. Skin penetration data may be expressed as % of dose and/or ug/cm².

### Example Combinations

1. A cosmetic skin care composition, comprising:
   (a) a peptide derivative having a formula: Rc-R₁-Gly-Pro-Gln-Gly-R₂,
wherein Rc is selected from the group of hydroxycinnamoyl, its derivative, and mixtures thereof,
wherein R₁ and R₂ are respectively from about 0 to about 8 amino acids, and wherein a total of R₁ and R₂ is 8 or less amino acids;
   (b) a fatty acid having from about 8 to about 22 carbon atoms; and
   (c) a dermatologically acceptable carrier.
2. The composition of the preceding feature 1, wherein R₁ and R₂ are respectively from about 0 to about 4 amino acids.
3. The composition of any of the preceding features, wherein a total of R₁ and R₂ is from about 0 to 8 amino acids, preferably from about 0 to 4 amino acids.
4. The composition of any of the preceding features, wherein a R₁ is zero and R₂ is from about 0 to 8 amino acids, preferably from about 0 to 4 amino acids.
5. The composition of any of the preceding features, wherein the R₂ is X or X-Gln, wherein X is Pro, Ile-Ala-Ser, or Pro-Ala-Ser.
6. The composition of any of the preceding features, wherein Rc is selected from the group consisting of caffeoyl, para-coumaroyl, feruloyl, and mixtures thereof.
7. The composition of any of the preceding features, wherein the peptide derivative is Caffeoyl Hexapeptide 9, wherein Rc is caffeoyl, R₁ is zero and R₂ is Pro-Gln.
8. The composition of any of the preceding features, wherein the fatty acid is selected from the group consisting of stearic acid, palmitic acid, and mixtures thereof, preferably wherein the fatty acid is a mixture of stearic acid and palmitic acid, more preferably wherein the fatty acid is a mixture of stearic acid and palmitic acid at a weight ratio of from about 3:7 to about 7:3.
9. The composition of any of the preceding features, wherein the peptide is present at a level of from about 0.0001% to about 5%, preferably from about 0.001% to about 1%, more preferably from about 0.003% to about 0.5%, by weight of the composition.
10. The composition of any of the preceding features, wherein the fatty acid is present at a level of from about 0.01% to about 10%, preferably from about 0.05% to about 5%, more preferably from about 0.05.% to about 1%, by weight of the composition.
11. The composition of any of the preceding features, wherein the peptide and the fatty acid are present at a ratio of from about 1:5 to about 1:1000, preferably from about 1:10 to about 1:100, more preferably from about 1:10 to about 1:50.
12. The composition of any of the preceding features, further comprising at least one additional ingredient selected from vitamins, minerals, other peptides, a saccharide, a botanical extract, sugar amines, sunscreen agents, oil control agents, flavonoid compounds, anti-oxidants, protease inhibitors, tyrosinase inhibitors, anti-inflammatory agents, moisturizing agents, exfoliating agents, skin lightening agents, anti-acne agents, anti-wrinkle agents, phytosterols, N-acyl amino acid compounds, antimicrobials, antifungals, pH adjustors, thickening agents, preservatives, and combinations thereof, preferably selected from the group consisting of a vitamin B3 compound, a vitamin A compound, a vitamin E compound, a saccharide, a botanical extract, other peptides, and mixtures thereof.
13. A non-therapeutic method of treating skin, comprising:
   (a) identifying a target portion of skin where treatment is desired; and
   (b) applying the skin care composition of any of the preceding claims to the target portion
   of skin over the course of a treatment period, and
preferably wherein the method improves penetration of the peptide into skin.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cosmetic skin care composition, comprising:
a) a peptide derivative having a formula: Rc-R₁-Gly-Pro-Gln-Gly-R₂,
wherein Rc is chosen from hydroxycinnamoyl, its derivative, or mixtures thereof, wherein
R₁ and R₂ are respectively from about 0 to about 8 amino acids, and wherein a total of R₁ and R₂ is 8 or less amino acids;
b) a fatty acid having from about 8 to about 22 carbon atoms; and
c) a dermatologically acceptable carrier.

2. The composition of claim 1, wherein R₁ is zero and R₂ is X or X-Gln, wherein X is Pro, Ile-Ala-Ser, or Pro-Ala-Ser.

3. The composition of claim 1, wherein Rc is selected from the group consisting of caffeoyl, para-coumaroyl, feruloyl, and mixtures thereof.

4. The composition of claim 1, wherein the peptide derivative is Caffeoyl Hexapeptide 9, wherein Rc is caffeoyl, R₁ is zero and R₂ is Pro-Gln.

5. The composition of claim 1, wherein the fatty acid is selected from the group consisting of stearic acid, palmitic acid, and mixtures thereof.

6. The composition of claim 5, wherein the fatty acid is a mixture of stearic acid and palmitic acid.

7. The composition of claim 6, wherein the fatty acid is a mixture of stearic acid and palmitic acid at a weight ratio of from about 3:7 to about 7:3.

8. The composition of claim 1, wherein the peptide is present at a level of from about 0.0001% to about 5% by weight of the composition.

9. The composition of claim 8, wherein the peptide is present at a level of from about 0.001% to about 1% by weight of the composition.

10. The composition of claim 9, wherein the peptide is present at a level of from about 0.003% to about 0.5% by weight of the composition.

11. The composition of claim 1, wherein the fatty acid is present at a level of from about 0.01% to about 10% by weight of the composition.

12. The composition of claim 11, wherein the fatty acid is present at a level of from about 0.05% to about 5% by weight of the composition.

13. The composition of claim 12, wherein the fatty acid is present at a level of from about 0.05% to about 1% by weight of the composition.

14. The composition of claim 1, wherein the peptide and the fatty acid are present at a ratio of from about 1:5 to about 1:1000.

15. The composition of claim 14, wherein the peptide and the fatty acid are present at a ratio of from about 1:10 to about 1:100.

16. The composition of claim 15, wherein the peptide and the fatty acid are present at a ratio of from about 1:10 to about 1:50.

17. The composition of claim 1, further comprising at least one additional ingredient chosen from vitamins, minerals, other peptides, a saccharide, a botanical extract, sugar amines, sunscreen agents, oil control agents, flavonoid compounds, anti-oxidants, protease inhibitors, tyrosinase inhibitors, anti-inflammatory agents, moisturizing agents, exfoliating agents, skin lightening agents, anti-acne agents, anti-wrinkle agents, phytosterols, N-acyl amino acid compounds, antimicrobials, antifungals, pH adjustors, thickening agents, preservatives, or mixtures thereof.

18. The composition of claim 17, wherein the additional ingredient is selected from the group consisting of a vitamin B3 compound, a vitamin A compound, a vitamin E compound, a saccharide, a botanical extract, other peptides, and mixtures thereof.

19. A non-therapeutic method of treating skin, comprising:
a) identifying a target portion of skin where treatment is desired; and
b) applying the skin care composition of claim 1 to the target portion of skin over the course of a treatment period.

20. The method of claim 19, wherein the method improves penetration of the peptide into skin.
